(19) United States
(12) Patent Application Publication
(10) Pub. No.: US 2018/0249776 A1
(43) Pub. Date: Sep. 6, 2018

(54) CLOTHING HAVING ANION-EMITTING SILICONE PATCH ATTACHED THERETO AND METHOD FOR MANUFACTURING SAME

(71) Applicants: Tae Gil Park, Gyeonggi-do (KR); GMK CO., LTD., Gyeonggi-do (KR); ENERSKIN INC., Seoul (KR)

(72) Inventor: Tae Gil Park, Gyeonggi-do (KR)

(21) Appl. No.: 15/760,379

(22) PCT Filed: Sep. 9, 2016
(86) PCT No.: PCT/KR2016/010216

§ 371 Date: Mar. 15, 2018

(30) Foreign Application Priority Data
Sep. 16, 2015 (KR) 10-2015-0131249

(51) Int. Cl. A41D 27/08; A41H 42/00; A61K 9/70; A61K 47/34; A61K 31/192; A61K 33/22; A61K 33/42; A61K 33/00; A61K 33/24

(52) U.S. Cl. CPC A41D 27/08; A41H 42/00; A61K 9/70; A61K 47/34; A41D 2400/32; A61K 33/22; A61K 33/42; A61K 33/00; A61K 33/24; A61K 31/192

(57) ABSTRACT

The present invention relates to clothing having an anion-emitting silicone patch attached thereto and a method for manufacturing same. The clothing having an anion-emitting silicone patch attached thereto according to the present invention comprises: clothing; and a silicone patch which is adhered to the clothing and emits anions, wherein the silicone patch comprises anion ore powder and is adhered to clothing by seamless welding at a temperature ranging from 170 to 180° C. under a pressure of 45 to 55 psi applied for 9 to 13 seconds. Accordingly, the present invention manufactures silicone patches using an anion-emitting material and adheres same to clothing, thereby, as a result of the anions, improving blood circulation in the human body and relieving fatigue, and enabling the patches to be firmly attached to the clothes by silicone. The patches exhibit excellent adhesiveness and washing fastness even after washing and remain steadily attached regardless of the passage of time, and the anion-emitting material is not easily removed, enabling the long, semi-permanent use thereof.

### TECHNICAL FIELD

The present invention relates to clothing having an anion-emitting silicone patch attached thereto and a method for manufacturing the same, and more specifically, to clothing having an anion-emitting silicone patch attached thereto, in which blood circulation in the human body is improved and fatigue is relieved by anions, and the patches are firmly attached to the clothes by silicone to allow long-term use of clothing without anion loss by manufacturing a silicone patch using an anion-emitting material and attaching the silicone patch to clothing, and a method for manufacturing the same.

### BACKGROUND ART

Generally, clothing has been developed as a medium for the desire of expression of the human along with the history of mankind, and has begun with the practicality of controlling heat or cold in response to climate changes or protecting the body from obstacles from the outside, and decorativeness and sociality have gradually been added thereto. Such changes and developments are, of course, important factors in promoting the development of clothing, aesthetically and scientifically, and materials are freely chosen according to purpose, and the creation of more beautiful and functional clothing has been continued.

Recently, clothing has been manufactured to be easy to work, and the proportion of clothes is gradually increasing as the tendency of clothing to be used for various purposes such as focusing on functional aspects or pursuing decorativeness is increasing.

In contemporary society, various functionalities are required for clothing, for example, clothing not only needs to suit the body shape because clothing expresses the inside, personality, and even social status of the human, but should also meet the desire for health as health issues such as the environment and stress socially emerged.

Further, in order to form patterns on clothing, the surface of clothing has been conventionally coated with PVC or PU as a main material, but the coating film of these materials was too thin to be actually applied to clothing, and the actual use thereof was not implemented very much due to many environmentally harmful aspects thereof.

However, when a pattern is formed on the surface of clothing by using a silicone resin as a main material, the thickness of the silicone resin coating film can be freely thinned or thickened, and the silicone resin does not cause environmental problems, and thus the use thereof has been gradually increasing.

The coating film in which the silicone resin is used as a main material should have a constant shape and have a suitable viscosity when cured by heat. The viscosity should be maintained within a certain range during the coating operation. If not, there arises a problem that the pattern of the coating becomes small and the shape thereof collapses when peeling off the coating from the surface of the clothing after the clothing is coated with the coating. Further, the pattern of the silicone resin coating film after the heating process may be cured in a form different from that originally designed, thereby deteriorating the marketability of the fabric or clothing.

Further, various ornaments for decorating clothing are provided according to the needs of various consumers. Among them, silicone-based ornaments are attracting attention because the silicone-based ornaments give a visual sense of depth and can be firmly attached to the surface of clothing.

These silicone technologies are being applied to the advertising, packaging and decorative industries. Among them, for the silicone to be applied as a decoration for clothing and the like, the regeneration efficiency of silicone should be high, and the adhesive strength and washing fastness (abrasion resistance) should be excellent even when washed, and the uniform attachment state should be maintained regardless of the elapsed time, due to the nature of clothing.

Conventionally, as a decoration for clothing, a method of dyeing or printing patterns or characters of a specific color on clothing during a process of manufacturing clothing, or a method of sewing a separate fabric has been used. However, the above-described method has low work efficiency, requires a separate manufacturing process, is not preferable in view of environmental friendliness due to the use of dyes and inks, and has reduced washing fastness during washing to have low marketability.

Accordingly, there has been a steady demand from the industry for ornaments for clothing in which silicone patterns of various shapes are formed in response to preferences of consumers. Further, in addition to the aesthetic functionality of silicone patterns, there has been a constant demand for ornaments for clothing with functional features considering health in the clothing that people wear at all times.

### DISCLOSURE

### Technical Problem

It is an object of the present invention to provide clothing having an anion-emitting silicone patch attached thereto, in which blood circulation in the human body is improved and fatigue is relieved by anions, and the patches are firmly attached to the clothes by silicone to allow long-term use of clothing without anion loss by manufacturing a silicone patch using an anion-emitting material and attaching the silicone patch to clothing, and a method for manufacturing the same.

It is another object of the present invention to provide clothing having an anion-emitting silicone patch attached thereto, in which the patches exhibit excellent adhesiveness and washing fastness even after washing and remain steadily attached regardless of the passage of time, and the anion-emitting material is not easily removed, enabling the long-term semi-pennanent use thereof, and a method for manufacturing the same.

However, the scope of the present invention is not limited to the above-described objects, and other unmentioned objects may be clearly understood by those skilled in the art from the following descriptions.

### Technical Solution

Clothing having an anion-emitting silicone patch attached thereto according to the present invention includes: clothing; and a silicone patch which is adhered to the clothing and emits anions, in which the silicone patch includes anion ore powder, and the silicone patch is adhered to clothing by seamless welding at a temperature ranging from 170 to 180° C. under a pressure of 45 to 55 psi applied for 9 to 13 seconds.

The anion ore powder may be formed of a mixed powder including tourmaline powder, monazite powder, elvan powder, and germanium powder, and may further include ketoprofen. The tourmaline powder, the monazite powder, the elvan powder, and the germanium powder may be pulverized and powdered into particles with a particle size of 400 to 500 mesh. The tourmaline powder, the monazite powder, the elvan powder and the germanium powder may form a mixed powder including the tourmaline powder at 25 to 35 parts by weight, the monazite powder at 5 to 20 parts by weight, the elvan powder at 25 to 35 parts by weight, and the germanium powder at 25 to 35 parts by weight. The mixed powder may be included at 5 to 10 parts by weight based on 100 parts by weight of the total silicone resin, and the ketoprofen may be included at 0.5 to 1.5 parts by weight based on 100 parts by weight of the total tourmaline powder, the monazite powder, the elvan stone powder and the germanium powder.

The number of anions emitted by the anion ore powder contained in the silicone patch is configured to be in the range of 1000 to 2000 ions/cc.

Further, a method of manufacturing clothing having an anion-emitting silicone patch attached thereto according to the present invention includes: a liquid silicone resin preparation step (S100) of preparing a liquid silicone resin; an additive adding step (S200) of adding anion ore powder to the liquid silicone resin and mixing; an adhesion enhancing agent and curing agent addition step (S300) of adding an adhesive enhancing agent and a curing agent to the liquid silicone resin mixed with the additive and mixing; a clothing fabric printing step (S400) of printing the liquid silicone resin mixed with the additive, the adhesion enhancing agent and the curing agent on a clothing fabric; a rolling step (S500) of expanding the liquid silicone resin printed on the clothing fabric to a predetermined thickness and width; a curing step (S600) of introducing the silicone expanded to a predetermined thickness and width into a heating furnace and curing; and a silicone patch preparation step (S700) of separating and cooling the cured clothing fabric to complete a silicone patch attached to clothing, in which, in the additive adding step (S200), the anion ore powder is formed of a mixed powder including tourmaline powder, monazite powder, elvan powder, and germanium powder, and further includes ketoprofen, the tourmaline powder, the monazite powder, the elvan powder, and the germanium powder are pulverized and powdered into particles with a particle size of 400 to 500 mesh, the tourmaline powder, the monazite powder, the elvan powder and the germanium powder form a mixed powder including the tourmaline powder at 25 to 35 parts by weight, the monazite powder at 5 to 20 parts by weight, the elvan powder at 25 to 35 parts by weight, and the germanium powder at 25 to 35 parts by weight, the mixed powder is included at 5 to 10 parts by weight based on 100 parts by weight of the total silicone resin, and the ketoprofen is included at 0.5 to 1.5 parts by weight based on 100 parts by weight of the total tourmaline powder, the monazite powder, the elvan stone powder and the germanium powder.

In the additive adding step (S200), the mixed powder may be included at 5 to 10 parts by weight based on 100 parts by weight of the total liquid silicone resin, and in the adhesion enhancing agent and curing agent addition step (S300), the adhesion enhancing agent may be included at 2 to 4 parts by weight based on 100 parts by weight of the total liquid silicone resin, and the curing agent may be included at 1 to 2 parts by weight based on 100 parts by weight of the total liquid silicone resin.

In the rolling step (S500), the printed liquid silicone may be adhered by applying a pressure of 45 to 55 psi at a temperature ranging from 170 to 180° C. for 9 to 13 seconds.

The curing step (S600) may be performed for 3 to 5 minutes after maintaining a curing furnace at a temperature of 65 to 75° C.

The details of other embodiments are included in the detailed description of the present invention.

### Advantageous Effects

According to the clothing having an anion-emitting silicone patch attached thereto of the present invention, blood circulation in the human body is improved and fatigue is relieved by anions, and the patches are firmly attached to the clothes by silicone to allow long-term use of clothing without anion loss by manufacturing a silicone patch using an anion-emitting material and attaching the silicone patch to the clothing.

Further, according to the clothing having an anion-emitting silicone patch attached thereto of the present invention, the patches exhibit excellent adhesiveness and washing fastness even after washing and remain steadily attached regardless of the passage of time, and the anion-emitting material is not easily removed, enabling the long-term semi-permanent use thereof.

It will be apparent to those skilled in the art that the embodiments in accordance with the present invention can provide various effects not specifically mentioned.

### DESCRIPTION OF DRAWINGS

FIG. 1 is a flowchart for explaining a method of manufacturing clothing having an anion-emitting silicone patch attached thereto according to the present invention.

FIG. 2 is a photograph showing a capillary blood flow before wearing clothing having an anion-emitting silicone patch attached thereto, which is manufactured according to the present invention.

FIG. 3 is a photograph showing a capillary blood flow after wearing clothing having an anion-emitting silicone patch attached thereto, which is manufactured according to the present invention.

FIGS. 4 and 5 are views showing a part of meridian points of the human body.

### BEST MODE OF THE INVENTION

The advantages and characteristic features, and the method for achieving the advantages of the present invention will be clarified by the embodiments described hereinafter in detail and the attached drawings. However, the present invention is not limited to the embodiments provided herein but can be implemented in various ways. Rather, the embodiments set forth and described hereinafter are provided to help the present invention to be understood easily and clearly, and to provide the persons skilled in the art with sufficient information related with the present invention.

The terms used in this description are adopted only to describe the specific embodiment, and are not intended to limit the scope of the present invention. The singular forms in this description include plural forms unless the context clearly states otherwise.

Unless otherwise defined herein, the terms including the scientific terms used in this description have the meanings that are generally understood by those skilled in the art. The terms having the meanings as defined in common dictionaries should be interpreted as the meanings in the context of the art, and should not be interpreted in an ideal or excessively formal meaning unless clearly defined otherwise in this description.

Hereinafter, clothing having an anion-emitting silicone patch attached thereto according to the present invention will be described in detail.

According to the clothing having an anion-emitting silicone patch attached thereto of the present invention, by manufacturing a silicone patch using an anion-emitting material and attaching the silicone patch to clothing, blood circulation in the human body is improved and fatigue is relieved by anions, and the patches are firmly attached to the clothes by silicone to allow long-term use of clothing without anion loss.

The clothing having an anion-emitting silicone patch attached thereto according to the present invention includes: clothing; and a silicone patch which is adhered to the clothing and emits anions.

The clothing having an anion-emitting silicone patch attached thereto according to the present invention may be any of various types of known clothing. For example, clothing such as sportswear such as ski suits, outdoor wear, innerwear, underwear and the like as well as various products such as sports bands or compression bands, hats, shawls and the like may be used.

Further, the silicone patch which will be described below may be adhered to the clothing according to the present invention by seamless welding using a dedicated press machine. Here, the silicone patch may be adhered to the clothing by applying a pressure of 45 to 55 psi at a temperature ranging from 170 to 180° C. for 9 to 13 seconds. In the present invention, when adhesion of the silicone patch is performed at a condition below the above-described lower limit, the silicone patch is hardly attached to the clothing. When adhesion of the silicone patch is performed at a condition above the above-described lower limit, an excessive pressure or temperature may cause damage to clothing or silicone patches.

The silicone patch is adhered to the clothing, and may be adhered by seamless welding. In the present invention, the silicone patch includes anion ore powder.

The anion ore powder is ore powder that emits anions. The anions enhance the ionization rate of calcium, sodium, potassium and the like, which are minerals in the blood, to purify the blood through the progress of alkalization, and generates substances such as endorphins and enkephalins to increase the ionization rate of calcium and sodium in the serum, allowing blood purification, and recovery form fatigue and recovery of physical strength and activating cells of an area causing pain to be healthy, and thereby the pain can be alleviated and the blood can be purified by electrical material exchange in a cell membrane.

In the present invention, the anion ore powder may be mixed with the silicone resin after pulverizing ore that emits anions into a powder form. Examples of the ore powder which emits anions include a mixed powder of tourmaline powder, monazite powder, elvan powder, and germanium powder.

In the present invention, the tourmaline powder, the monazite powder, the elvan powder, and the germanium powder may be pulverized and powdered into particles with a particle size of 400 to 500 mesh. The silicone patch may be formed by mixing a mixed powder including the tourmaline powder at 25 to 35 parts by weight, the monazite powder at 5 to 20 parts by weight, the elvan powder at 25 to 35 parts by weight, and the germanium powder at 25 to 35 parts by weight with a silicone resin and molding. Here, the mixed powder formed by mixing the tourmaline powder, the monazite powder, the elvan powder, and the germanium powder may be included at 5 to 10 parts by weight based on 100 parts by weight of the total silicone resin.

In the present invention, the anion ore powder may emit anions through the tourmaline powder, the monazite powder, the elvan powder, and the germanium powder, and the number of anions emitted by the anion ore powder may be configured to be in the range of 1000 to 2000 ions/cc.

The tourmaline has a current which is the same as a weak current of 0.06 mA required to maintain the life of human beings, and has new-far-infrared emissivity (0.923), pyroelectric (PYRO) and piezoelectric (PIEZO) properties. Such tourmaline not only emits a large amount of anions, but also converts electromagnetic waves into good energy. Thus, the tourmaline has effects of promoting metabolism, relieving pain, promoting blood circulation and hemokinesis, fast recovery from diseases, anti-aging, warming, sweating and the like.

The monazite is a mineral belonging to monoclinic phosphate minerals of cerium rare earth elements, and may form granular or monocrystalline crystals to form granules and masses. The monazite contained in the anion ore powder layer includes 50.2% silicon dioxide, 14.3% alumina oxide, 3.1% sodium oxide, 2.3% magnesium oxide, 2.3% potassium oxide, 11% calcium oxide, 1.1% phosphorus oxide and 13% iron oxide, and not only emits a large amount of infrared rays, but can also emit a larger amount of anions compared to other minerals.

The elvan serves to discharge harmful substances in the body to the outside of the body through an ion exchange action in which mineral elements such as calcium (Ca), sodium (Na), potassium (K) and magnesium (Mg) beneficial to the human body are discharged, and serves to prevent the colonization of Escherichia coli, Pseudomonas aeruginosa, Staphylococcus aureus and molds by an antimicrobial action, decompose and remove odors, and is particularly effective against skin diseases, and can increase immunity and greatly help in wound healing.

The germanium activates the biological current flowing in the human body when the germanium comes into contact with the human body to improve blood circulation, thereby relieving symptoms such as knotted muscles, fatigue, pain and the like, inhibits the accumulation of fatigue substances and also enhances the natural healing power inherent in humans, and has the effect of adjusting body balance such as relieving muscular fatigue in sports that entail a lot of movements, pain of muscles and joints accumulated in the daily life, shoulder stiffness, backaches, neurological diseases and the like, or alleviation of mental fatigue caused by stress.

In the present invention, the anion ore powder may further include ketoprofen. The ketoprofen is used as a nonsteroidal anti-inflammatory drug, and the ketoprofen the ketoprofen does not cause a blood concentration thereof to rapidly increase and the drug can be rapidly and continuously delivered to the skin tissue. In the present invention, the ketoprofen may be included at 0.5 to 1.5 parts by weight based on 100 parts by weight of the total anion ore powder.

Hereinafter, a method of manufacturing clothing having an anion-emitting silicone patch attached thereto according to the present invention will be described in detail with reference to the attached drawings.

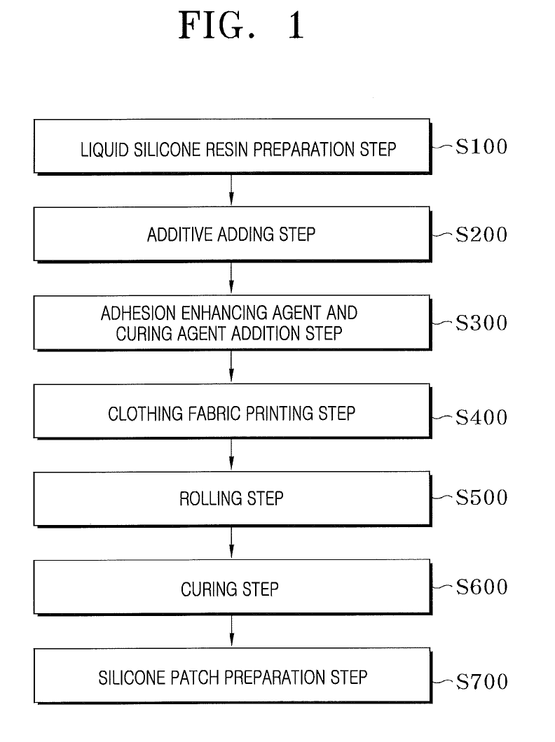

FIG. 1 is a flowchart for explaining a method of manufacturing clothing having an anion-emitting silicone patch attached thereto according to the present invention.

The method of manufacturing clothing having an anion-emitting silicone patch attached thereto according to the present invention includes a liquid silicone resin preparation step (S100); an additive adding step (S200); an adhesion enhancing agent and curing agent addition step (S300); a clothing fabric printing step (S400); a rolling step (S500); a curing step (S600); and a silicone patch preparation step (S700).

1. Liquid Silicone Resin Preparation Step (S100)

The liquid silicone resin preparation step (S100) is a step of heating a silicone crystal to liquefy the crystal to prepare a liquid silicone resin.

In the present invention, the liquid silicone resin preparation step (S100) will be described by way of example in which the silicone crystal is liquefied by heating, but the present invention is not limited thereto, and the liquid silicone which has already been liquefied may be used to prepare clothing having an anion-emitting silicone patch attached thereto according to the present invention.

In the present invention, an example in which a patch is formed using the liquid silicone prepared through the liquid silicone resin preparation step (S100) is described, but the technical idea of the present invention is not necessarily limited thereto, and those skilled in the art will recognize that the same functions and effects can be obtained by preparing a patch using liquid urethane or liquid rubber in addition to the liquid silicone.

2. Additive Adding Step (S200)

The additive adding step (S200) is a step of adding the anion ore powder to the liquid silicone resin and mixing, and the anion ore powder may be a mixed powder formed by mixing tourmaline powder, monazite powder, elvan powder, and germanium powder. The mixed powder may be included in an amount of 5 to 10 parts by weight based on 100 parts by weight of the total liquid silicone resin.

In the additive adding step (S200), an example in which the anionic ore powder is mixed with the liquid silicone resin is described, but liquid urethane or liquid rubber may be added to improve the physical properties and functionality of the silicone patch to be prepared, and inks and pigments may be added to express various colors and textures. Since such a configuration is well known to those skilled in the art, a detailed description thereof will be omitted.

Further, ketoprofen may be further included in the additive adding step (S200). The ketoprofen is used as a nonsteroidal anti-inflammatory drug, and the drug can be delivered quickly and continuously to skin tissue without a rapid increase in a blood concentration thereof In the present invention, the ketoprofen may be used in an amount of 0.5 to 1.5 parts by weight based on 100 parts by weight of the total anion ore powder.

3. Adhesion Enhancing Agent and Curing Agent Addition Step (S300);

An adhesion enhancing agent and curing agent addition step (S300) is a step of adding an adhesion enhancing agent and a curing agent to the liquid silicone mixed with the additive, and mixing.

In the present invention, the adhesion enhancing agent and the curing agent are separately added to the liquid silicone resin without being added in the additive adding step in which the anion ore powder is added to the liquid silicone resin and mixed, so that the liquid silicone resin is uniformly mixed with the anion ore powder, pigments and the like in the additive adding step.

That is, in the present invention, since the adhesion enhancing agent and the curing agent have high viscosity, the anion ore powder, pigments and the like may not be uniformly mixed with the liquid silicone resin. Therefore, the adhesion enhancing agent and the curing agent may be added after uniformly mixing the anion ore powder, pigments or the like with the liquid silicone resin in the additive adding step. In the present invention, the adhesion enhancing agent may be included at 2 to 4 parts by weight based on 100 parts by weight of the total liquid silicone resin, and the curing agent may be included at 1 to 2 parts by weight based on 100 parts by weight of the total liquid silicone resin.

4. Clothing Fabric Printing Step (S400)

The clothing fabric printing step (S400) is a step of printing the liquid silicone mixed with the additive, the adhesion enhancing agent and the curing agent on a clothing fabric. For example, the clothing fabric printing step may be performed using a silk screen printing method after the liquid silicone mixed with the additive, the adhesion enhancing agent and the curing agent is put into a coating material feeding cylinder of a silk printing machine.

5. Rolling Step (S500)

The rolling step (S500) is a step of expanding the liquid silicone printed on the clothing fabric to a predetermined thickness and width.

In the present invention, the rolling step (S500) may be a step of printing the liquid silicone on the clothing fabric and then pressing a printed material using a roller or a dedicated press so that the liquid silicone is adhered by seamless welding.

In the rolling step (S500), the printed liquid silicone may be adhered to clothing by applying a pressure of 45 to 55 psi at a temperature of 170 to 180° C. for 9 to 13 seconds. In the present invention, when the rolling step (S500) is performed at a condition below the above-described lower limit, silicone is hardly adhered to the clothing. When the rolling step is performed at a condition above the above-described upper limit, an excessive pressure or temperature may cause damage to clothing or silicone.

6. Curing Step (S600)

The curing step (S600) is a step of introducing the silicone expanded to a predetermined thickness and width after the rolling step (500) into a heating furnace and curing.

In the present invention, the curing step (S600) may be performed for 3 to 5 minutes after a curing furnace is maintained at a temperature of 65 to 75° C. The time and temperature of the curing process (S600) may be variously changed according to the thickness and size of the silicone patch to be prepared.

7. Silicone Patch Preparation Step (S700).

The silicone patch preparation step (S700) is a step of separating and cooling the cured clothing fabric to complete a silicone patch attached to clothing.

In the present invention, since the silicone patch is adhered to clothing using the anion ore powder as described above, blood circulation in the human body may be improved by anions, and the silicone patch can be used for a long time without anion loss by adhering the silicone patch to clothing by seamless welding.

In an embodiment of the present invention, the silicone patch is prepared using an anion ore material which emits anions and ketoprofen which is a pain relief medicine, and is adhered to clothing and used, and thereby anions can improve the blood circulation of the human body and alleviate fatigue, ketoprofen can relieve pain in the muscles, as well as exert a beneficial influence on body fluids, cells and nervous system to recover from body fatigue. In the present invention, the silicone patch may be configured to be adhered to the position of the clothing corresponding to a meridian point of the human body.

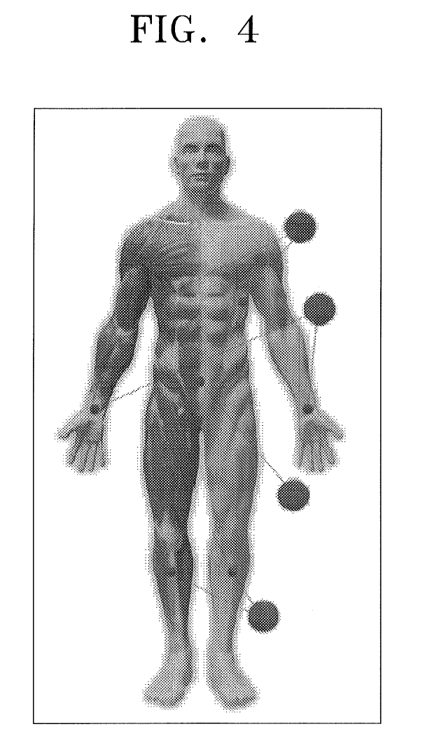

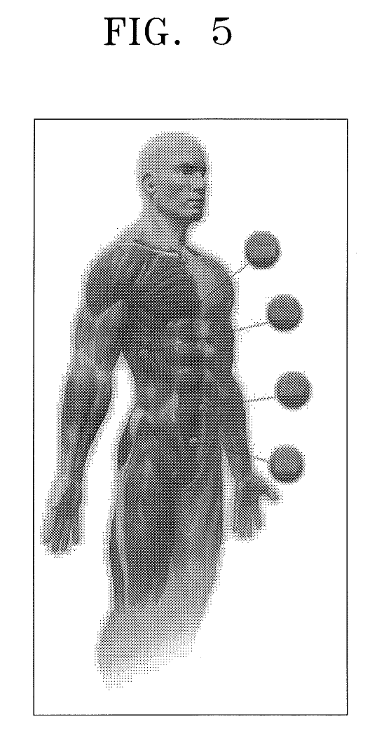

FIGS. 4 and 5 are views showing a part of meridian points of the human body. The clothing having an anion-emitting silicone patch attached thereto according to the present invention can significantly increase the effect of the silicone patch by configuring the silicone patch to correspond to a meridian point of the human body as shown in FIGS. 4 and 5.

### MODE OF THE INVENTION

Hereinafter, an example of the method of manufacturing clothing having an anion-emitting silicone patch attached thereto according to the present invention will be described in detail.

### EXAMPLE

First, 8 parts by weight of anionic ore powder was added to 100 parts by weight of a total liquid silicone resin and mixed. Here, the anion ore powder was formed by mixing tourmaline powder, monazite powder, elvan powder, and germanium powder in a weight ratio of 26:20:27:26, and ketoprofen was further mixed so as to be included at 1 part by weight based on 100 parts by weight of the total anion ore powder.

Next, liquid urethane, rubber, inks and pigments were added to the liquid silicone resin mixed with the anion ore powder, and then mixed uniformly. Thereafter, an adhesion enhancing agent and a curing agent were further added thereto.

Then, the liquid silicone mixed with the additive, the adhesion enhancing agent and the curing agent was put into a coating material feeding cylinder of a silk printing machine and then the mixture was printed using a silk screen printing method.

Subsequently, the silicone-printed clothing was pressed using a dedicated press machine and cured at a temperature of 70° C. for 3 minutes, and the cured clothing was separated and cooled to prepare clothing having an anion-emitting silicone patch attached thereto according to the present invention.

The clothing having an anion-emitting silicone patch attached thereto prepared according to the example was worn by subjects, and the capillary blood flow before and after wearing the clothing was measured.

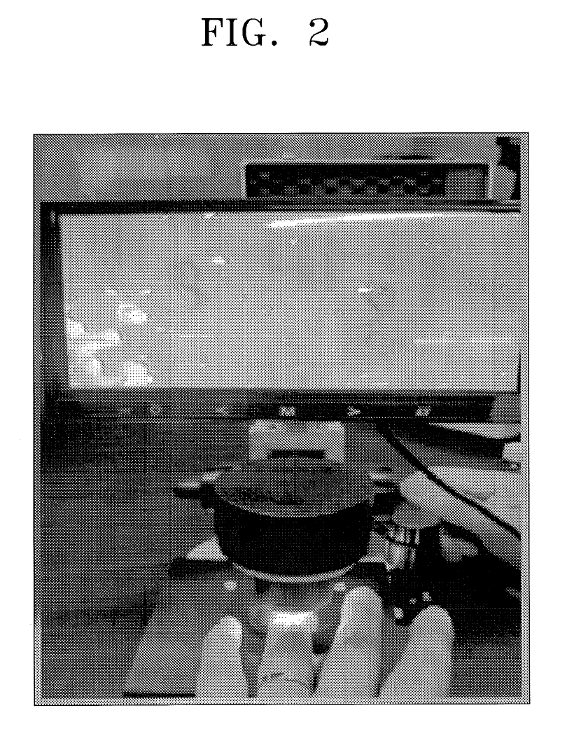

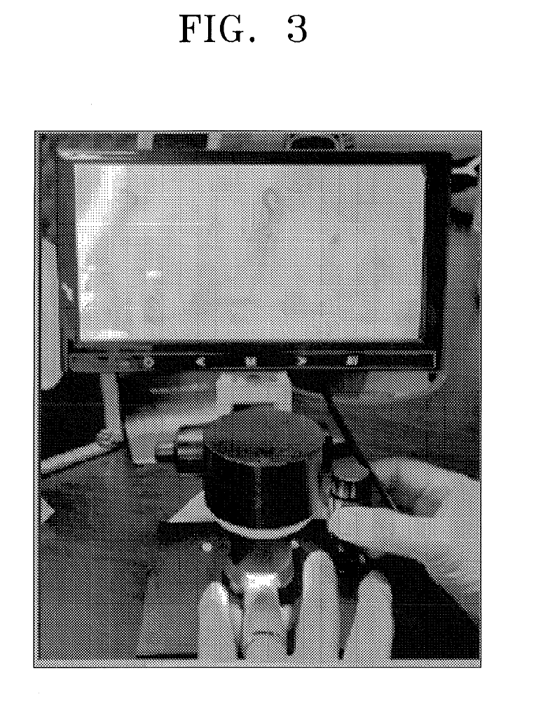

FIG. 2 is a view showing a capillary blood flow before wearing the clothing having an anion-emitting silicone patch attached thereto, which is manufactured according to the present invention, and FIG. 3 is a view showing a capillary blood flow after wearing the clothing having an anion-emitting silicone patch attached thereto, which is manufactured according to the present invention.

Referring to FIGS. 2 and 3, it can be seen that the capillary blood flow after wearing the clothing having an anion-emitting silicone patch attached thereto according to the present invention is active, which is because the anion ore powder contained in the silicone patch improves the blood circulation of the human body by emitting anions, thereby alleviating fatigue.

Although embodiments have been described with reference to a number of illustrative embodiments thereof, it should be understood that numerous other modifications and embodiments can be devised by those skilled in the art that will fall within the spirit and scope of the invention as defined by the following claims.

1. Clothing having an anion-emitting silicone patch attached thereto, comprising:
clothing; and
a silicone patch adhered to the clothing wherein the silicone patch emits anions,
wherein the silicone patch includes anion ore powder,
the silicone patch is adhered to the clothing by seamless welding at a temperature ranging from 170 to 180° C. under a pressure of 45 to 55 psi applied for 9 to 13 seconds.

2. The clothing according to claim 1, wherein the anion ore powder is formed of a mixed powder including tourmaline powder, monazite powder, elvan powder, and germanium powder, and further includes ketoprofen, and the tourmaline powder, the monazite powder, the elvan powder, and the germanium powder are pulverized and powdered into particles with a particle size of 400 to 500 mesh, the tourmaline powder, the monazite powder, the elvan powder and the germanium powder form a mixed powder including the tourmaline powder at 25 to 35 parts by weight, the monazite powder at 5 to 20 parts by weight, the elvan powder at 25 to 35 parts by weight, and the germanium powder at 25 to 35 parts by weight, the mixed powder is included at 5 to 10 parts by weight based on 100 parts by weight of the total silicone resin, and the ketoprofen is included at 0.5 to 1.5 parts by weight based on 100 parts by weight of the total tourmaline powder, the monazite powder, the elvan stone powder and the germanium powder.

3. The clothing according to claim 2, wherein the number of anions emitted by the anion ore powder contained in the silicone patch is configured to be in the range of 1000 to 2000 ions/cc.

4. A method of manufacturing clothing having an anion-emitting silicone patch attached thereto, comprising:
a liquid silicone resin preparation step of preparing a liquid silicone resin (S100);
an additive adding step of adding anion ore powder to the liquid silicone resin and mixing (S200);
an adhesion enhancing agent and curing agent addition step of adding an adhesive enhancing agent and a curing agent to the liquid silicone resin mixed with the additive and mixing (S300);
a clothing fabric printing step of printing the liquid silicone resin mixed with the additive, the adhesion enhancing agent and the curing agent on a clothing fabric (S400);
a rolling step of expanding the liquid silicone resin printed on the clothing fabric to a predetermined thickness and width (S500);
a curing step of introducing the silicone expanded to a predetermined thickness and width into a heating furnace and curing (S600); and
a silicone patch preparation step of separating and cooling the cured clothing fabric to complete a silicone patch attached to clothing (S700),
wherein, in the additive adding step (S200), the anion ore powder is formed of a mixed powder including tourmaline powder, monazite powder, elvan powder, and germanium powder, and further includes ketoprofen,
the tourmaline powder, the monazite powder, the elvan powder, and the germanium powder are pulverized and powdered into particles with a particle size of 400 to 500 mesh, the tourmaline powder, the monazite powder, the elvan powder and the germanium powder form a mixed powder including the tourmaline powder at 25 to 35 parts by weight, the monazite powder at 5 to 20 parts by weight, the elvan powder at 25 to 35 parts by weight, and the germanium powder at 25 to 35 parts by weight, the mixed powder is included at 5 to 10 parts by weight based on 100 parts by weight of the total silicone resin, and
the ketoprofen is included at 0.5 to 1.5 parts by weight based on 100 parts by weight of the total tourmaline powder, the monazite powder, the elvan stone powder and the germanium powder.

5. The method according to claim 4, wherein, in the additive adding step (S200), the mixed powder is included at 5 to 10 parts by weight based on 100 parts by weight of the total liquid silicone resin, and
in the adhesion enhancing agent and curing agent addition step (S300), the adhesion enhancing agent is included at 2 to 4 parts by weight based on 100 parts by weight of the total liquid silicone resin, and the curing agent is included at 1 to 2 parts by weight based on 100 parts by weight of the total liquid silicone resin.

6. The method according to claim 4, wherein, in the rolling step (S500), the printed liquid silicone is adhered by applying a pressure of 45 to 55 psi at a temperature ranging from 170 to 180° C. for 9 to 13 seconds.

7. The method according to claim 4, wherein the curing step (S600) is performed for 3 to 5 minutes after maintaining a curing furnace at a temperature of 65 to 75° C.
